(19) United States
(12) Patent Application Publication
(10) Pub. No.: US 2022/0175984 A1
(43) Pub. Date: Jun. 9, 2022

(54) DISINFECTION DEVICE

(71) Applicants: AXIS LIGHTING INC., Lasalle (CA); STERILUMEN, INC., Tarrytown, NY (US)

(72) Inventors: Howard YAPHE, Lasalle (CA); Andrew MILES, Lasalle (CA); Nathanel TITANE, Lasalle (CA); Hristea MIHALCEA, Lasalle (CA); Donaki SIMMONS, Tarrytown, NY (US)

(21) Appl. No.: 17/114,447

(22) Filed: Dec. 7, 2020

(51) Int. Cl. A61L 2/10

(52) U.S. Cl. CPC A61L 2/10; A61L 2202/15; A61L 2202/14; A61L 2202/11

(57) ABSTRACT

Disclosed is a disinfection device for controllably emitting and directing disinfecting UV energy to at least one elongate first target location and at least one elongate second target location in a user space. The disinfection device comprises a housing structure mountable on a support structure and having an outer boundary. The outer boundary is configured to provide a first array of outputs to emit and direct disinfecting UV energy to the at least one first target location, a second array of outputs to deliver directed disinfecting UV energy to the at least one second target location.

### FIELD OF THE DISCLOSURE

The present disclosure relates to disinfection devices and disinfection systems using disinfection devices and associated structures, for use in interior or exterior user locations in a user space for directed emissions of UV energy to disinfect contact surfaces therein.

### BACKGROUND

The COVID-19 pandemic of 2020 has reinforced the need for improved control of pathogens in locations of user interaction that have user contact surfaces (or tough points), that is specific locations where people make contact with the same location, such as a tap in a bathroom, or a button on a parking meter or kiosk, as examples. Usually, this involves a manual cleaning to sterilize these kinds of surfaces. However, the degree of disinfection relies, in all cases, on the care and attention of the person performing the task.

There remains a need to improve modes of disinfecting surfaces, by way of devices that may be integrated into fixtures adjacent to, or providing, user contact surfaces.

### SUMMARY

An aspect may provide a disinfection device for controllably emitting and directing disinfecting UV energy to at least one first linear target location and at least one second linear target location in a user space, comprising a linear housing structure mountable on a support structure and having an outer boundary. The outer boundary may be configured to provide:
a first linear array of outputs to deliver directed disinfecting UV energy to the at least one first linear target location, with each output of the first array thereof configured to receive directed disinfecting UV energy from one instance of a first array of disinfecting UV energy directors, with each disinfecting director of the first array thereof configured to receive disinfecting UV energy from one instance of a first array of disinfecting UV energy emitters; and
a second linear array of outputs to deliver directed disinfecting UV energy to the at least one second linear target location, with each output of the second array thereof configured to receive directed disinfecting UV energy from one instance of a second array of disinfecting UV energy directors, with each disinfecting director of the second array thereof configured to receive disinfecting UV energy from one instance of a second array of disinfecting UV energy emitters.

Another aspect may provide a disinfection device for controllably emitting and directing disinfecting UV energy to at least one elongate first target location and at least one second target location in a user space. The device may comprise a housing structure mountable on a support structure and having an outer boundary, wherein the outer boundary may be configured to provide:
a first array of outputs to deliver directed disinfecting UV energy to the at least one first target location, with each output of the first array thereof configured to receive directed disinfecting UV energy from one instance of a first array of disinfecting UV energy directors, with each disinfecting director of the first array thereof configured to receive disinfecting UV energy from one instance of a first array of disinfecting UV energy emitters; and
a second array of outputs to deliver directed disinfecting UV energy to the at least one second target location, with each output of the second array thereof configured to receive directed disinfecting UV energy from one instance of a second array of disinfecting UV energy directors, with each disinfecting director of the second array thereof configured to receive disinfecting UV energy from one instance of a second array of disinfecting UV energy emitters.

Another aspect may provide a disinfection device for controllably emitting and directing disinfecting UV energy to at least one first target location and at least one second target location in a user space. The device may comprise a linear housing structure mountable on or in at least one support structure and having an outer boundary, wherein the outer boundary is configured to provide:
at least one output configured:
   to receive a first directed disinfecting UV energy from at least one instance of a first array of disinfecting UV energy directors, with each UV energy disinfecting director of the first array thereof configured to receive a first disinfecting UV energy from one instance of a first array of disinfecting UV energy emitters, and to deliver the first directed disinfecting UV energy to the at least one first target location; and
   to receive a second directed disinfecting UV energy from at least one instance of a second array of disinfecting UV energy directors, with each disinfecting UV energy director of the second array thereof configured to receive a second disinfecting UV energy from one instance of a second array of disinfecting UV energy emitters, and to deliver the second directed disinfecting UV energy to the at least one second target location.

Another aspect may provide a disinfection device for controllably emitting and directing disinfecting UV energy to at least one first proximal target location defined in a first plane, and at least one second distal target location defined in a second plane, in a user space. The device may comprise a housing structure mountable on or in at least one support structure and having an outer boundary, wherein the outer boundary is configured to provide:
at least one output configured:
   to receive a first directed disinfecting UV energy from a first array of at least one first disinfecting UV engine to deliver the first directed disinfecting UV energy to the at least one first proximal target location; and
   to receive a second directed disinfecting UV energy from a second array of second disinfecting UV engines to deliver the second directed disinfecting UV energy to the at least one second proximal target location.

### BRIEF DESCRIPTION OF THE FIGURES

Several exemplary embodiments of the present disclosure will be provided, by way of examples only, with reference to the appended drawings, wherein:

FIG. 1 is perspective view of a disinfection device shown schematically in an operable configuration;

FIG. 2 is a perspective assembly view of the disinfection device of FIG. 1;

FIG. 3 is a side assembly view and FIG. 6 is a side schematic view of the disinfection device of FIG. 2;

FIGS. 4, 5 and 6 are schematic cross sectional views of the disinfection device taken on lines 4-4, 5-5 and 6-6 respectively of FIG. 2;

FIGS. 7 to 9 are front and rear perspective views of another disinfection device;

FIG. 10 is a right hand side elevation of the device of FIG. 7, with the left hand side elevation being a mirror thereof and omitted;

FIG. 11 is a part assembly perspective sectional view taken along the break line of FIG. 7;

FIG. 12 is another sectional view according to FIG. 11 schematically illustrating first and second two target locations;

FIG. 12a is a magnified fragmentary part assembly perspective sectional view of a portion of the device shown in FIG. 11;

FIG. 13 is a perspective view of a housing structure segment of the device of FIG. 7;

FIG. 14 is a perspective sectional view of the housing structure segment taken on line 14-14 of FIG. 21;

FIG. 15 is a perspective sectional view of the housing structure segment taken through the break line of FIG. 14;

FIGS. 16, 17, 18 and 19 are top plan, bottom plan, front elevation and rear elevation views of the device of FIG. 7;

FIGS. 20 and 21 are upper front perspective and lower rear perspective views of the housing structure segment of the device of FIG. 13;

FIGS. 22, 23, 24 and 25 are top plan, bottom plan, front elevation and rear elevation views of the housing structure segment of the device of FIG. 13;

FIGS. 26, 27 and 28, are upper right front perspective, lower rear perspective, and upper left front perspective views of a support structure of the device of FIG. 7; and

FIGS. 29, 30, 31 and 32 are top plan, front elevation, bottom plan, and rear elevation views of the support structure of FIG. 26.

The broken lines used in the figures are provided for purposes of illustrating portions of the design that include environment. Further, the subject matter herein includes any of the present figures with any solid line therein replaced by a broken or stippled line, or vice versa. Thus, any such replacement is not new matter.

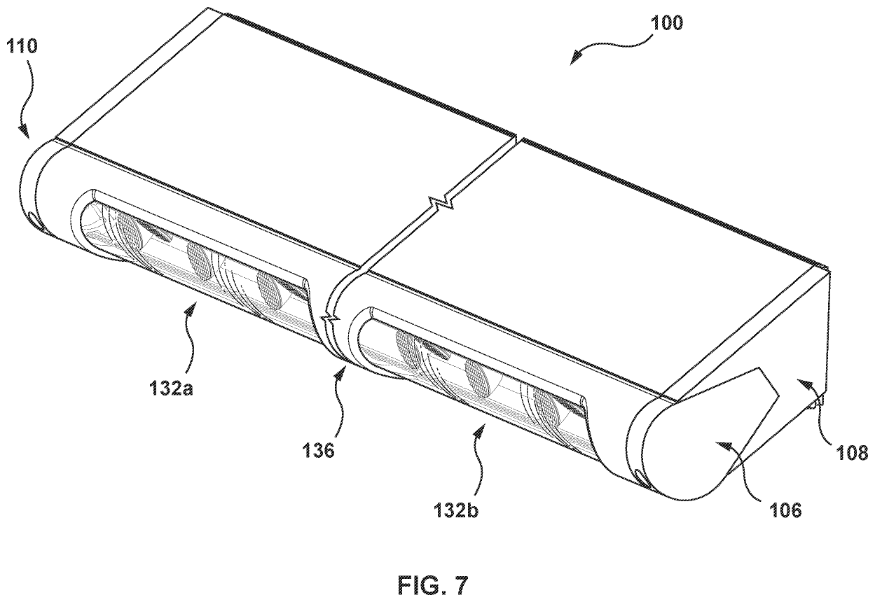

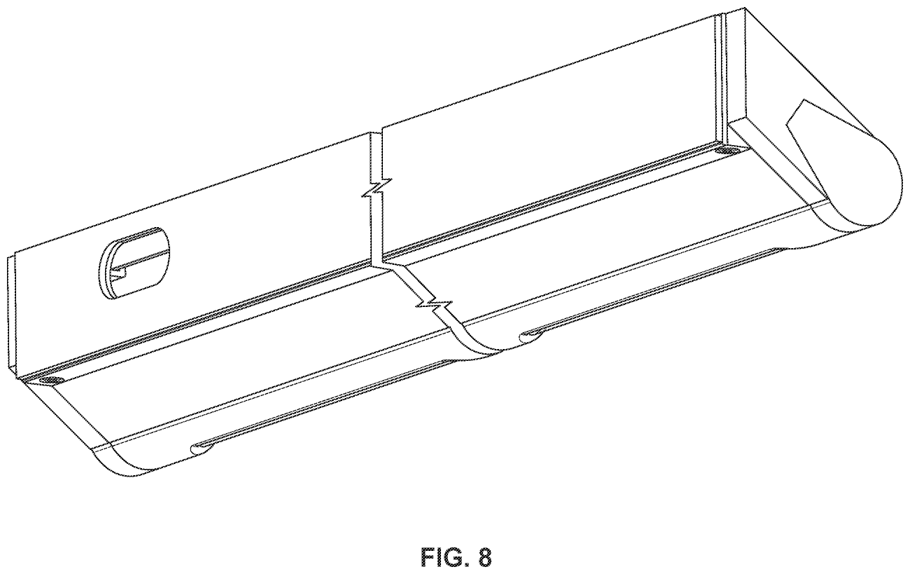

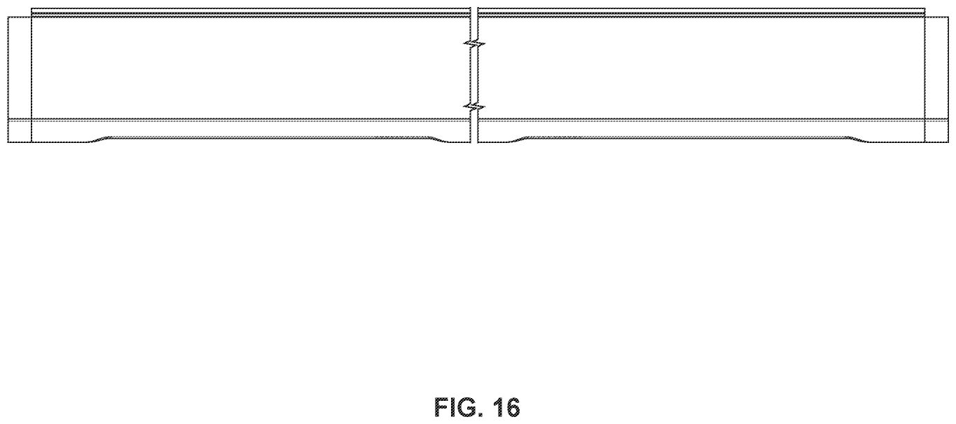

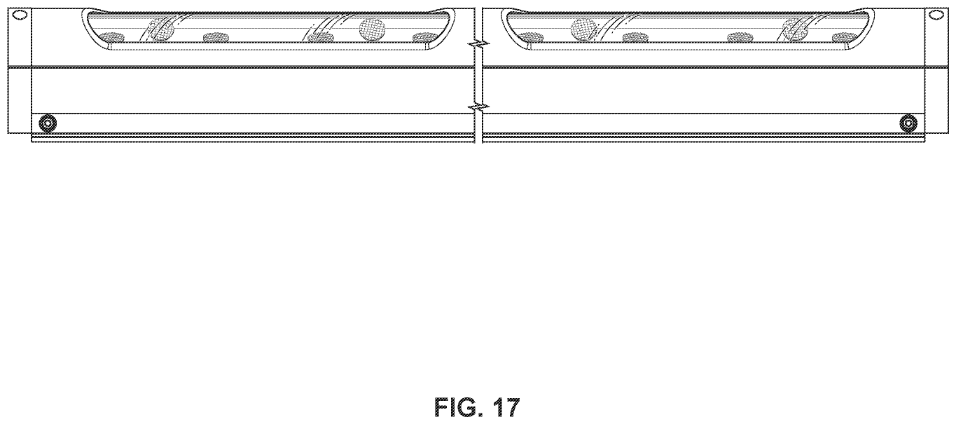

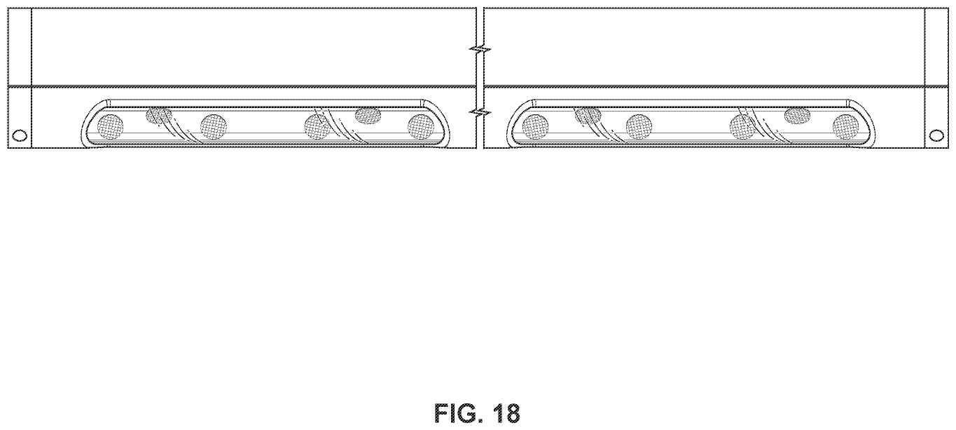

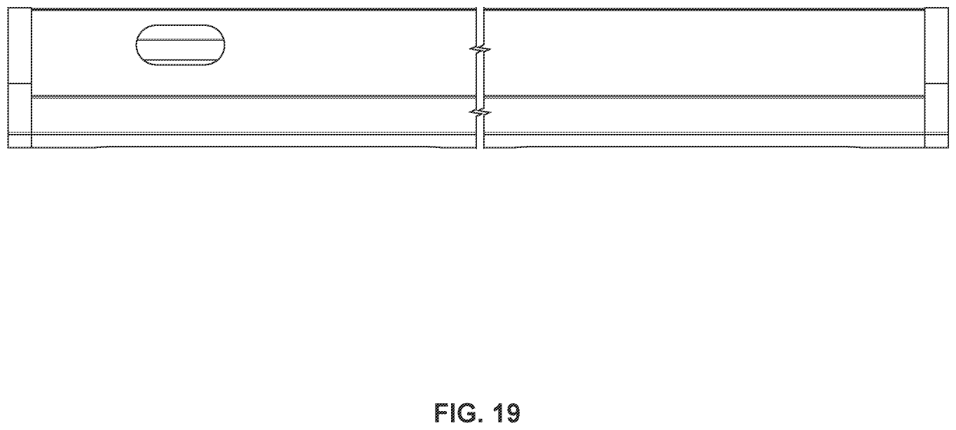

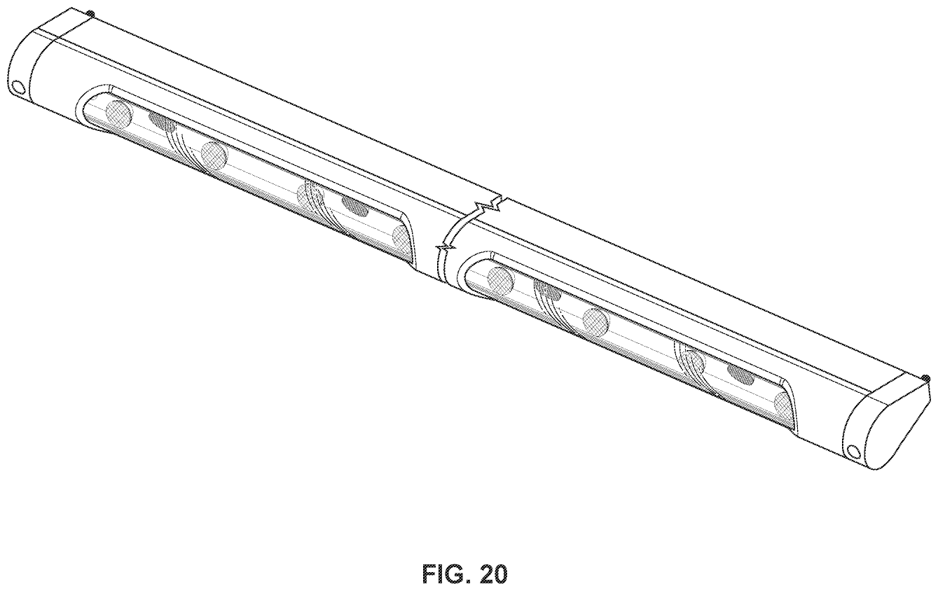

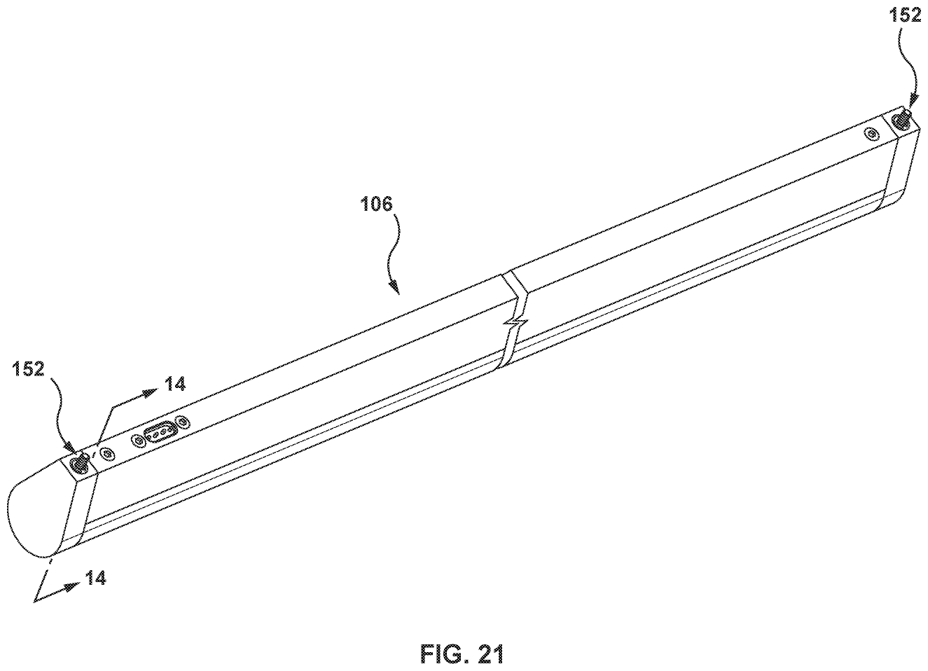

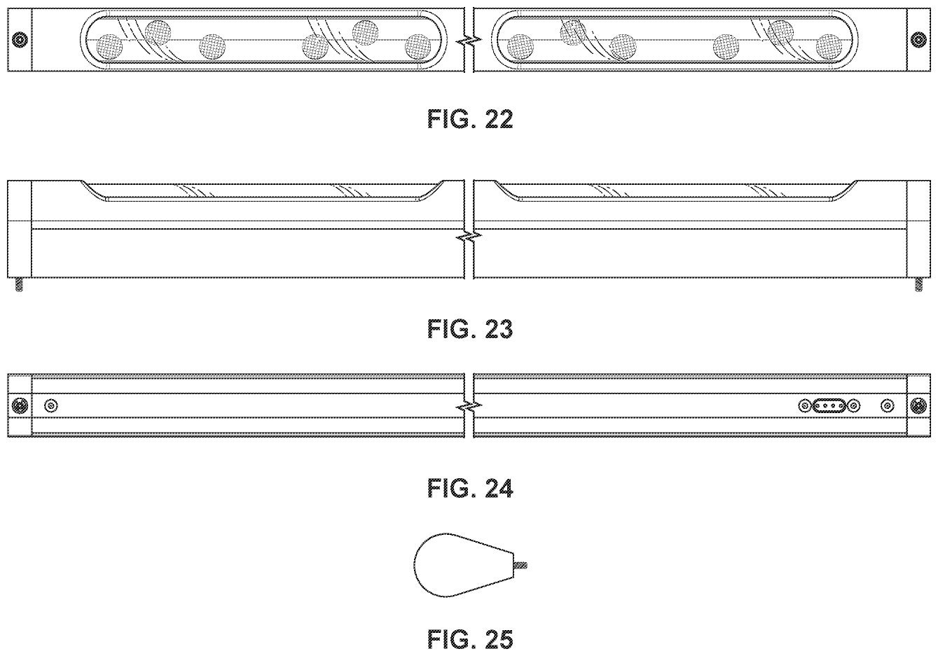

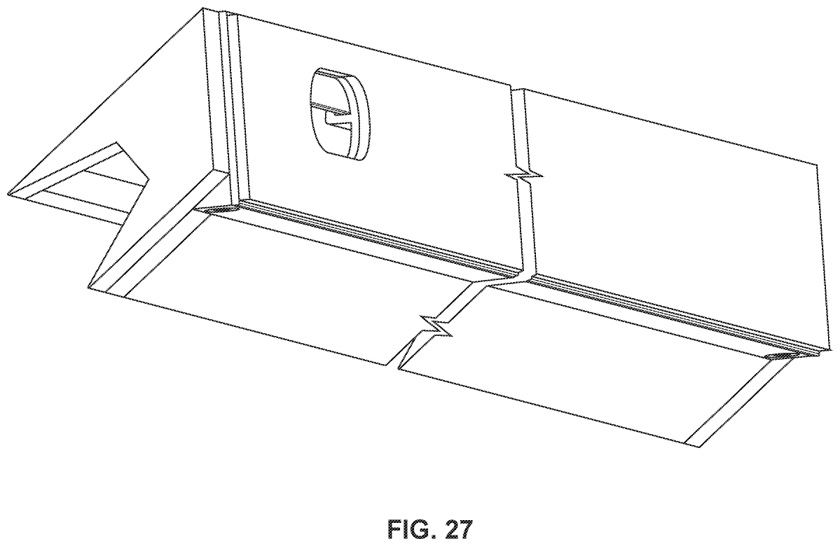

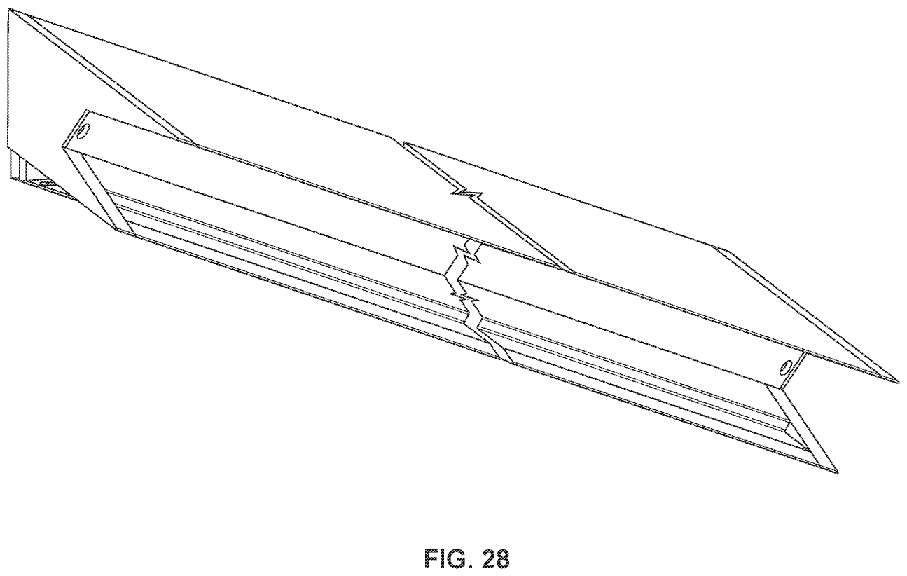

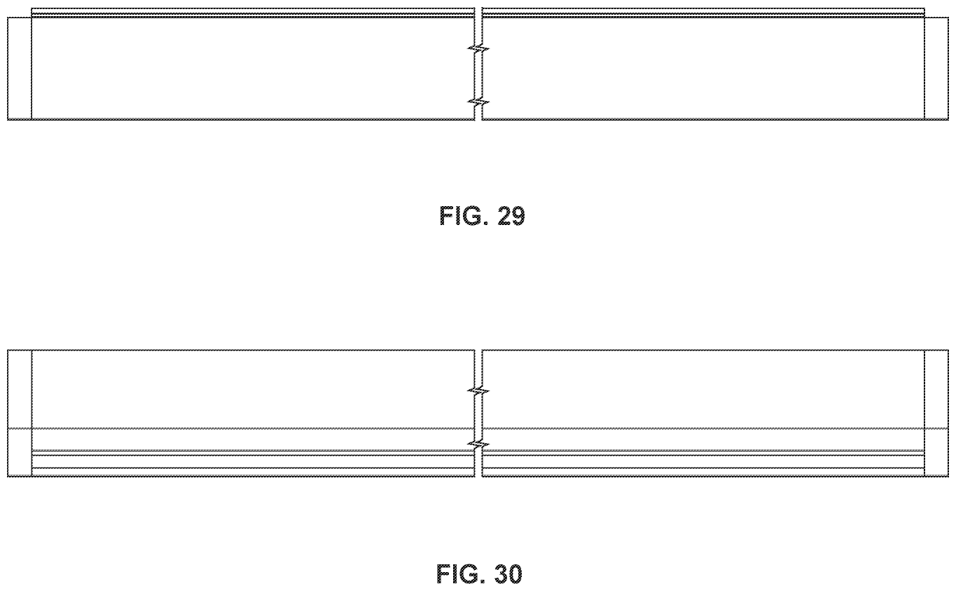

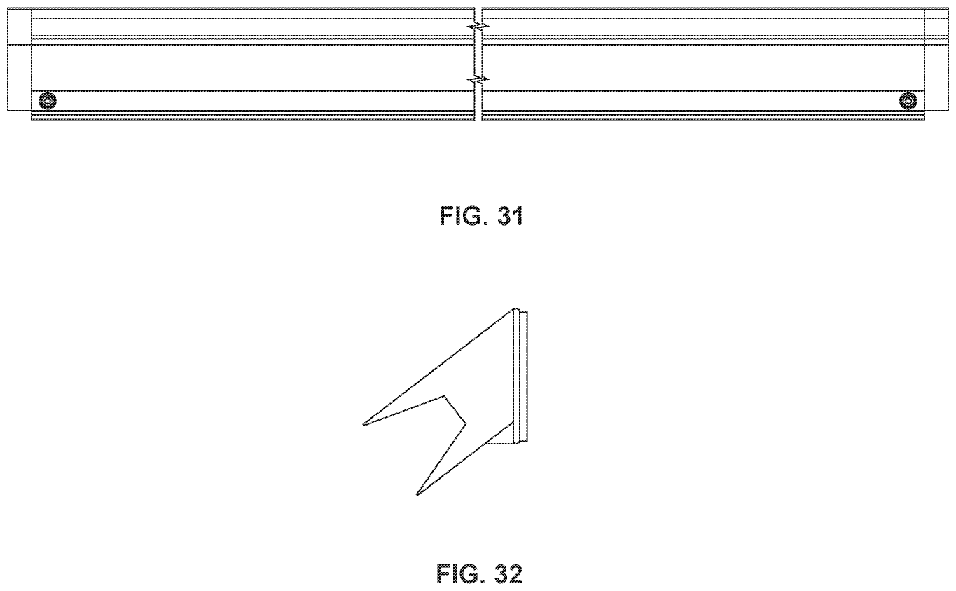

The disinfection device is shown in FIGS. 7-9, 13, 14, 16-24 and 26-31, with symbolic break lines along its length. The appearance of a portion of the luminaire structure between the break lines does not form part of the design. Any specific dimension of the article associated with the break lines is not claimed and is to be broadly interpreted. The subject matter herein includes any of the present figures with any such symbolic break lines removed, or with one or more further symbolic break lines added. Thus, any removal or addition of one or more any such symbolic break lines is not new matter. Further, the subject matter herein includes any of the present figures with any solid line therein replaced by a broken or stippled line, or vice versa.

### DETAILED DESCRIPTION

It should be understood that the invention is not limited in its application to the details of construction and the arrangement of components set forth in the following description or illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical, mechanical or other connections or couplings. The terms upper, lower, and vertical are intended for operative context only and are not necessarily intended to limit the invention only to those configurations or orientations. In any instance in which the disclosure refers to a single instance of an element, example embodiments may include a multiple of such elements. The term “at least one” in reference to any element is not intended to force an interpretation on any other reference elsewhere in the disclosure to a single instance of an element to mean only one such instance of the element. Furthermore, and as described in subsequent paragraphs, the specific mechanical and/or other configurations illustrated in the drawings are intended to exemplify embodiments of the invention. However, other alternative mechanical and/or other configurations are possible which are considered to be within the teachings of the instant disclosure. Furthermore, any one element, feature, structure, function, of any aspect and/or example embodiment described in the present disclosure including the figures, clauses and/or claims herein, may itself be claimed on its own or be combined with any one or more elements, features, structures, functions, and/or steps from the same or any other aspects and/or example embodiments described in the present disclosure including the figures, clauses and/and claims herein.

The term “UV LED” or variations thereof used herein may include, but not be limited to, semiconductor, electroluminescent, organic, polymeric or other material based ultraviolet energy sources, such as those which commonly known as generally as light emitting diodes, as well as polymeric and other such energy sources, equivalents and variants thereof, which emit energy at frequencies known to kill at least some viruses, bacteria and/or other pathogens where such pathogens have been made available for testing. The UV LED may be configured to emit UV energy in the range of frequencies including but not strictly bounded by UV-C: 200 to 280 nm: UV-B: 280 to 315 nm and UV-A: 315 to 400 nm.

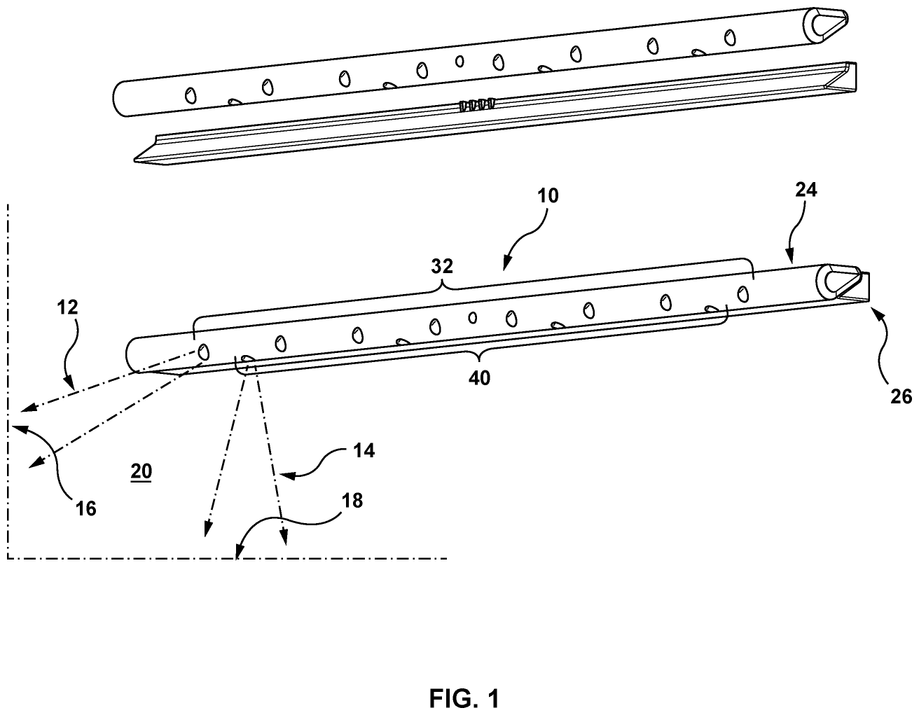

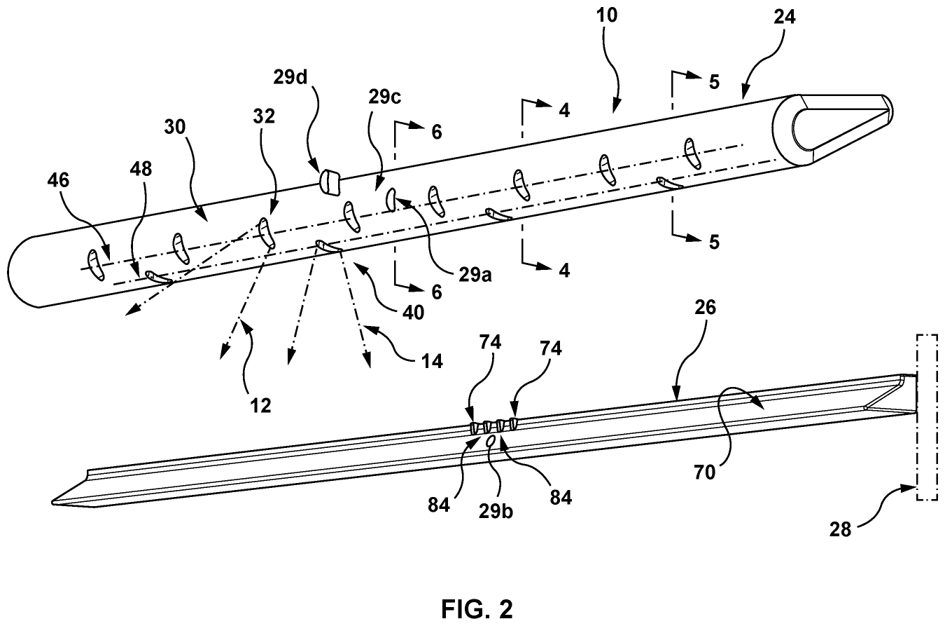

Referring to the FIG. 1 and subsequent figures, there is provided a disinfection device 10 for controllably emitting and directing UV energy, such as shown at 12, 14, to at least one target location, such as first target location 16, which can be an elongate first linear target location 16 and at least one second target location, such as second target location 18 which can be an elongate second linear location 18 at one or more designated common sites in an interior (or exterior) user space 20 such as a a sink, the walls, counter, and other surfaces surrounding and/or near the sink area as one example. The disinfection device 10 has a linear housing structure, which can be a linear housing structure 24 mountable on at least one support structure, which can be a linear support structure 26, which may be provided with at least one surface for abutting an appropriate mounting surface 28 (FIG. 2), such as on a lower surface of a mirror, or above a key pad on a kiosk, among other possible contact surfaces in user spaces.

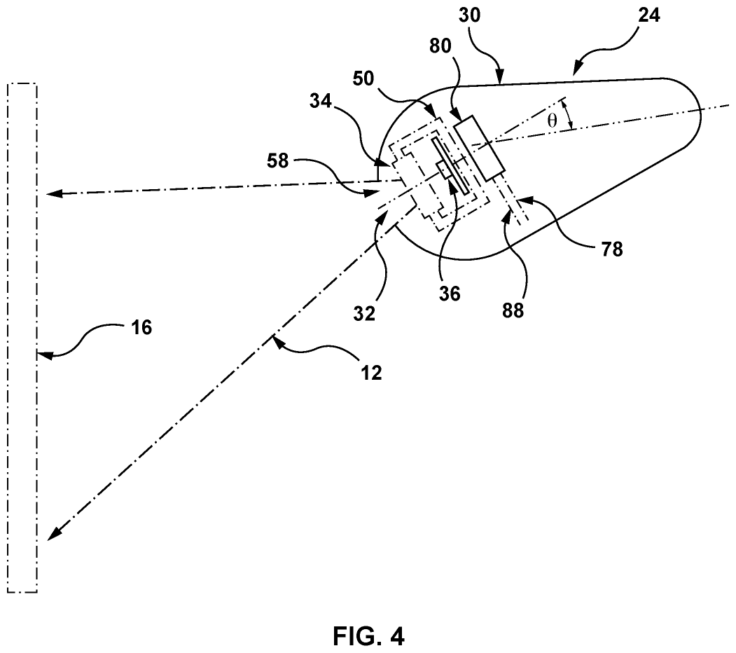

As shown in FIG. 4, the disinfection device 10 may be provided with an outer boundary 30 that may be configured to provide a first array of outputs shown at 32 in a generally linear orientation of first linear array of outputs 32 to deliver directed UV energy 12 at a first angle θ, relative to a reference axis of the linear housing structure 24, to the at least one first linear target location 16. Each output of the first array 32 may be configured to receive directed UV energy from one instance of a first array of UV energy directors, or first directors, shown at 34, such as a lens or other focusing or directing optic. Each first director of the first array 34 may be configured to receive UV energy from one instance of a first array of UV LEDs 36.

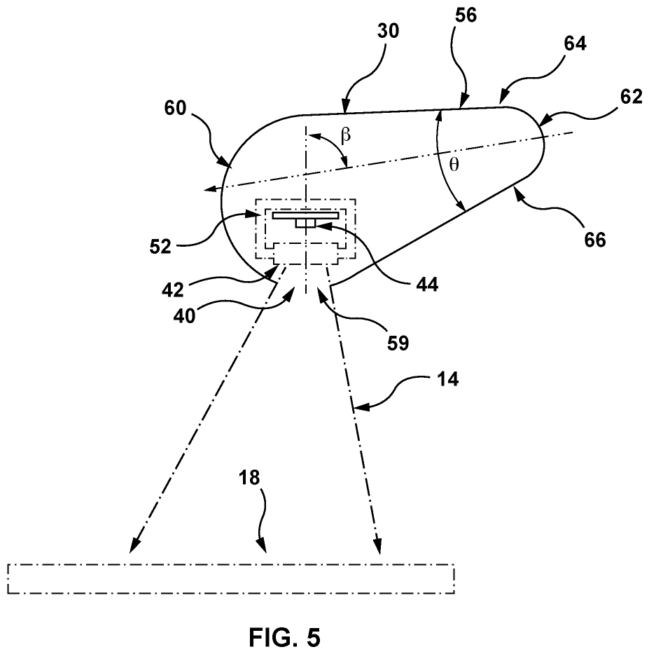

Referring to FIG. 5, the outer boundary 30 may be configured to provide a second array of outputs 40 in a generally linear orientation of second linear array of outputs 40 to deliver directed UV energy at a second angle θ to the at least one second linear target location 18. Each output of the second array 40 may be configured to receive directed UV energy from one instance of a second array of UV energy directors, or second directors, shown at 42 in FIG. 5. Each UV energy director of the second array 42 may be configured to receive UV energy from one instance of a second array of UV LEDs 44.

Referring to FIG. 2, the first array 32 and second array 40 can be seen to extend along respective parallel axes 46, 48 and such axes 46, 48 may be substantially parallel or substantially in common.

Referring to FIGS. 4 and 5, each instance of the UV energy directors of the first array 34, and the corresponding instance of the UV LEDs of the first array 36 may be positioned at a first mounting structure 50, such as a linear mounting structure 50, in the linear housing structure 24. Meanwhile, each instance of the UV energy directors of the second array 42, and the corresponding instance of the UV LEDs of the second array 44 may be positioned at a second mounting structure 52, such as a linear mounting structure 52, in the linear housing structure 24.

The outer boundary 30 of the linear housing structure 24 may be defined by an outer boundary wall 56, with each incidence of the first and second arrays of outputs 32 and 40 provided by corresponding passages 58, 59 through the boundary wall 56. The outer boundary wall 56 may have a continuous or discontinuous cross section. The outer boundary wall 56 may, for example, alternatively or in addition, have a curved, linear and/or curvilinear cross section.

Referring to FIG. 5, the outer boundary wall 56 may be provided with ellipsoidal (or circular) end regions 60, 62, with equal or unequal radii, with opposed wall sections extending therebetween, which may be oriented with an acute included angle α.

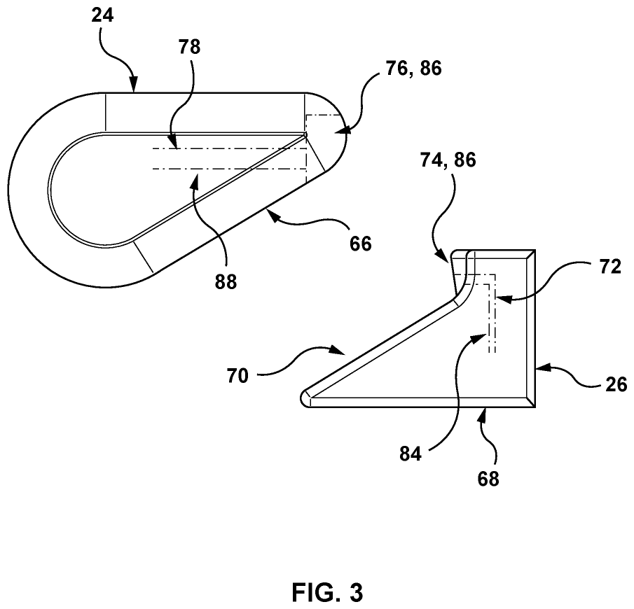

Referring to FIG. 3, the support structure 26 may include a base 68 with a support surface 70, which for illustration purposes may be seen to be inclined relative to the mounting structure 28 as seen in FIGS. 2 and 3, to engage a corresponding wall section 66.

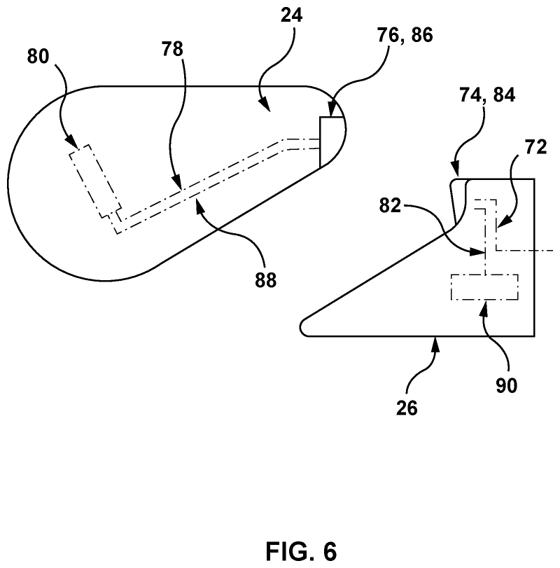

The support structure 26 may be further provided with a first power supply segment 72, while the linear housing structure 24 and support structure 26 are each configured with a first set of contacts 76, 74, respectively, for coupling the first power supply segment 72 to a second power supply segment 78 in the linear housing structure to energize the first and second arrays of UV energy emitters 36, 44. The second power supply segment 78 may include or be in communication with at least one driver 80, as shown in FIG. 4, to drive the UV LEDs, and which may be configured to exchange control data signals with a controller 90 (FIG. 6) networked therewith. In this case, while the driver 80 is shown in FIG. 4, as positioned adjacent the first linear mounting structure 50 and the first array of UV LEDs 36, the driver 80 may be configured to power both the first and second arrays of UV LEDs 36, 44.

Referring to FIG. 6, the support structure 26 may further include, as shown schematically, a first control data path segment 82, while the linear housing structure 24 and support structure 26 are each configured with a second set of contacts 84, 86 for coupling the first control data path segment 82 to a second control data path segment 88 in the linear housing structure 24 in communication with the driver 80. In this case, the controller 90 may be provided in the support structure 26, the linear housing structure 24, or be a remote controller located at a remote location.

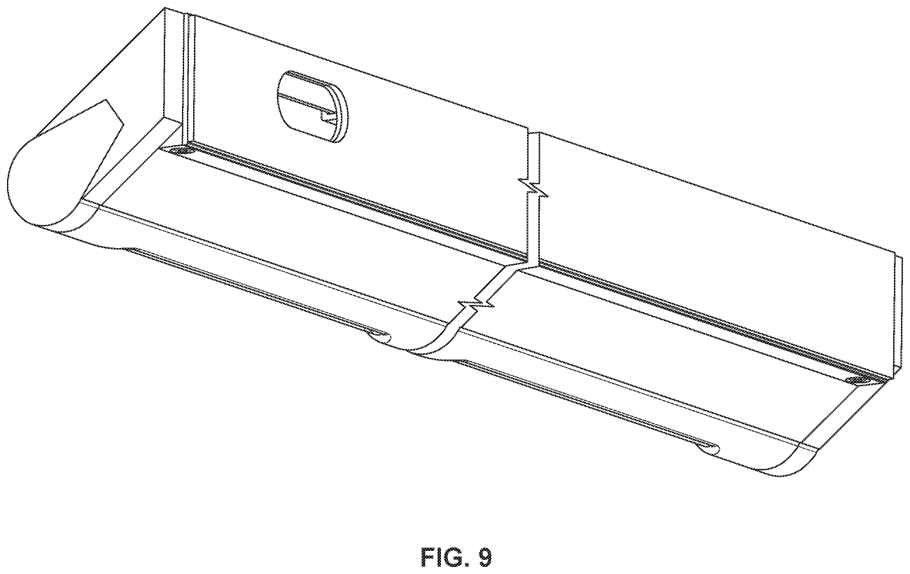

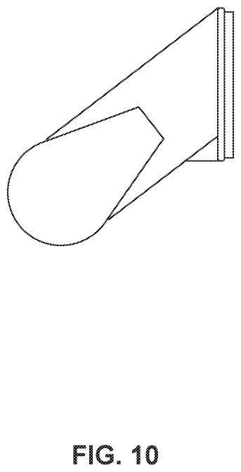

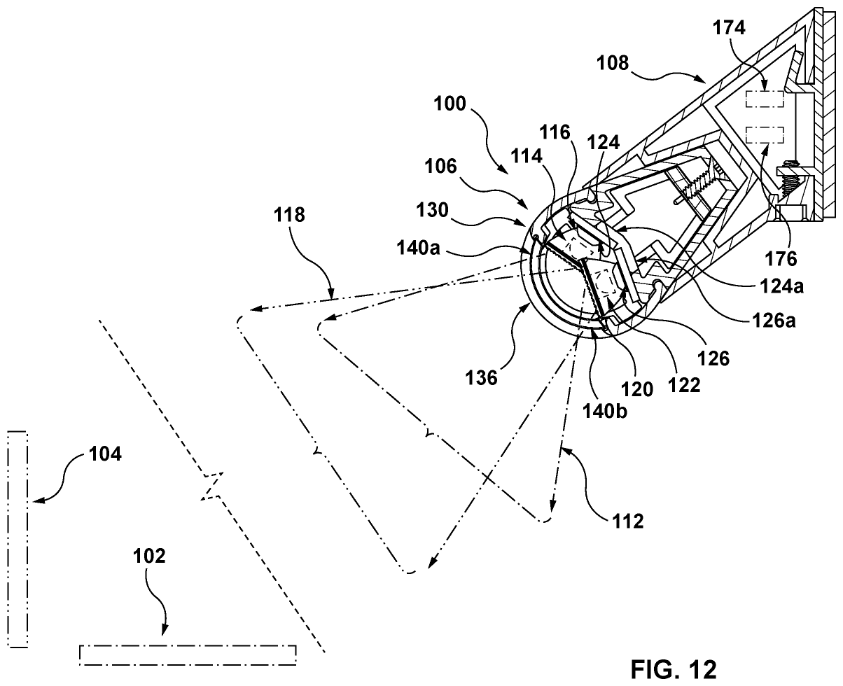

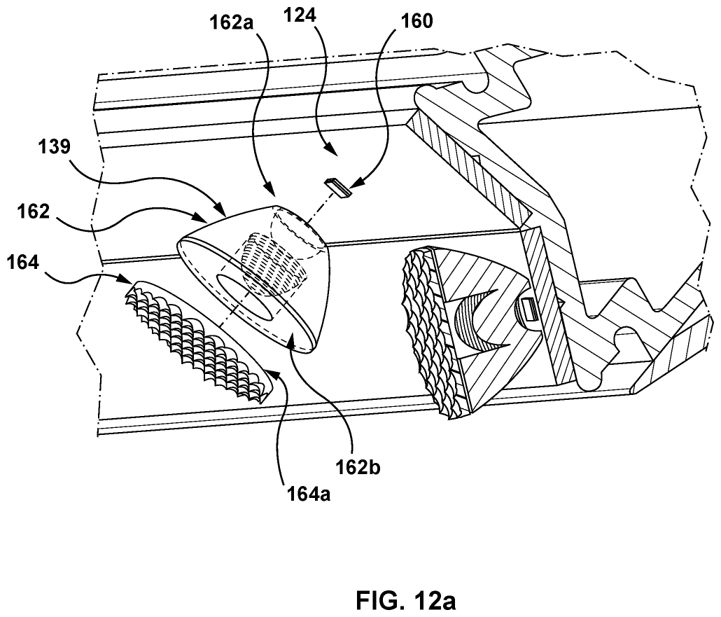

Another example embodiment of a disinfection device is shown at 100 in FIGS. 7 to 32 for controllably emitting and directing disinfecting UV energy to at least one first target location 102 (FIG. 12), which may be a linear first target location, and at least one second target location 104, which may be a linear second target location, in a user space. Referring to FIG. 7, the device 100 has a linear housing structure 106 mountable on or in at least one support structure 108 and has an outer boundary 110.

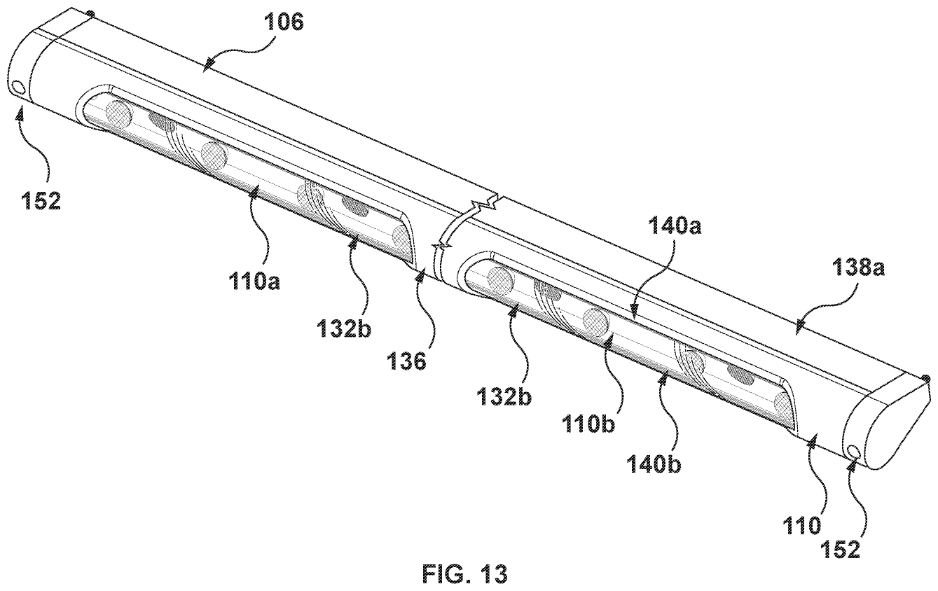

The linear housing structure 106 may be decoupled from the support structure 108, as shown in FIG. 13. The outer boundary 110 may be configured to provide at least one, in this case a pair of outputs 110a, 110b, which as shown in FIG. 13, may both be configured to receive a first directed disinfecting UV energy 112 (FIG. 12) from at least one instance of a first array of disinfecting UV energy directors shown at 114. Each UV energy disinfecting director 114 of the first array thereof may be configured to receive a first disinfecting UV energy from one instance of a corresponding first array of disinfecting UV energy emitters (in this example UV LEDs) shown at 116. The outputs 110a, 110b may thus be configured to deliver the first directed disinfecting UV energy 112 to the at least one first target location 102.

Likewise, both outputs 110a, 110b may be configured to receive a second directed disinfecting UV energy 118 from at least one instance of a second array of disinfecting UV energy directors shown at 120. Each disinfecting UV energy director 120 of the second array may be configured to receive a second disinfecting UV energy from one instance of a second array of disinfecting UV energy emitters (in this example UV LEDs) shown at 122. The outputs 110a, 110b may thus be configured to deliver the second directed disinfecting UV energy 118 to the at least one second linear target location 104. The first and second arrays, the housing structure and the support structures may be linear or nonlinear, while the housing structure may include more arrays as needed to as to provide the a multiple of arrays for the delivery of UV energy to at least the first and second target locations.

Referring to FIG. 12, each instance of the disinfecting UV energy directors 114 of the first array thereof, and the corresponding instance of the disinfecting UV energy emitters 116 of the first array thereof may be positioned adjacent a common location on a first linear mounting surface 124 in the linear housing structure 106.

As well, each instance of the disinfecting UV energy directors 120 of the second array thereof, and the corresponding instance of the disinfecting UV energy emitters 122 of the second array thereof may be positioned adjacent a common location on a second linear mounting surface 126 in the linear housing structure 106.

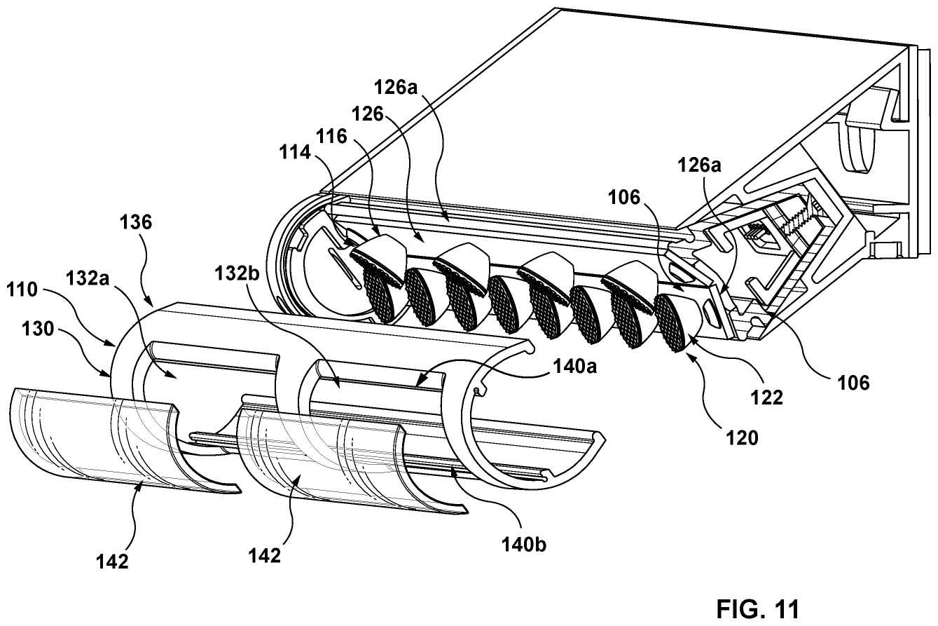

The first and second linear surfaces 124, 126 may be defined by corresponding first and second linear LED board structures 124a, 126a positioned in the linear housing structure 106. Referring to FIG. 11, the number of first UV energy directors 114 and first UV energy emitters 116 along the first linear mounting surface 124 may be fewer in number, than the number of second UV energy directors 120 and UV energy emitters 122 along the second linear mounting surface 126 (or vice versa), which enables a collective amount first directed disinfecting UV energy 112 delivered to the first target location 102 to be less than a collective amount second directed disinfecting UV energy 118 delivered to the second target location 104 (or vice versa).

Referring to FIG. 11, the outer boundary 110 of the linear housing structure 106 may be defined by an outer boundary wall 130, with each output 110a including a passage 132a, 132b through the boundary wall 110.

Referring to FIGS. 11, 13 and 14, the outer boundary wall 110 may be provided with at least one ellipsoidal end region 136, with a pair of opposed wall sections 138a, 138b extending therefrom. In this case, each passage 132a, 132b may extend through the at least one ellipsoidal end region 136 along a dimension substantially parallel to a central axis thereof.

Referring to FIG. 13, each passage 132a, 132b may be defined by a pair of opposed linear boundaries 140a, 140b that are spaced by an included radial angle δ, such as for example about 170 degrees, which may be selected according to a particular application.

In the case of FIG. 11, the first array of UV LEDs 116 can be seen to be interspersed between a corresponding pair of instances of the second array of UV LEDs 122 but alternate in such a way that there are alternating gaps where no such instance of the first array of UV LEDs 116 is provided. This may enable collective amounts of first and second directed disinfecting UV energy 112, 118, to be configured by the allocation of first UV energy directors and emitters 114, 116 and second UV energy directors and emitters 120, 122 as the case may be. For instance, different groupings of one or more UV energy directors and emitters may be provided in different combinations in the first and second arrays depending on the desired first and second directed disinfecting UV energies 112, 118. For instance, in a disinfecting device for use in a bathroom, mounted above a sink, a central grouping of second UV energy directors and emitters 120, 122 may be provided to deliver a greater level of the second directed disinfecting UV energy to a target location occupied by a faucet and facet knobs, compared with other neighboring areas, which may receive a substantially equivalent amount of second directed disinfecting UV energy as a first directed disinfecting UV energy being delivered to a wall surface above the sink.

In some example embodiments, the one or more groupings of second UV energy directors and second UV energy emitters 120, 122 in the second array may be higher in number, for example as shown in the example of FIG. 11, to take into account the greater distance of travel to the sink, while the one or more groupings of first UV energy directors and first UV energy emitters 114, 116 in the first array may be lower in number, for example as shown in the example of FIG. 11, to take into account the shorter distance of travel to a wall surface above the sink,

FIG. 12a shows a magnified view of UV engines 139 providing the first and second UV energy emitters 116 and 122 by way of UV LEDs 160, and the first and second UV energy directors 114 and 120 provided by a collimator structure 162 with a surface 162a to engage the first surface and second surfaces 124, 126, a diffuser structure 164 with a surface 164a to engage a corresponding surface 162b on the collimator structure 162.

Referring to FIG. 12, the first and second linear LED board structures 124a, 124b may each be configured to extend adjacent or along a corresponding linear boundary 140a, 140b, so as to cause to the first disinfecting UV energy 112 of the first array to cross or pass through the second directed disinfecting UV energy 118 of the second array (and vice versa).

Referring to FIG. 11, the disinfection device 100 may be further provided with a UV energy transmissive cover structure 142 to close each passage 132a, 132b. The cover structures 142 can be formed of UV energy transmissive materials which may include without limitation quartz and other materials or combinations thereof. In some example embodiments, the end region 136 may be planar or rectilinear, at least in part, to provide passages 132a, 132b such that they can receive cover structures that may be flat, rectilinear and/or planar at least in part.

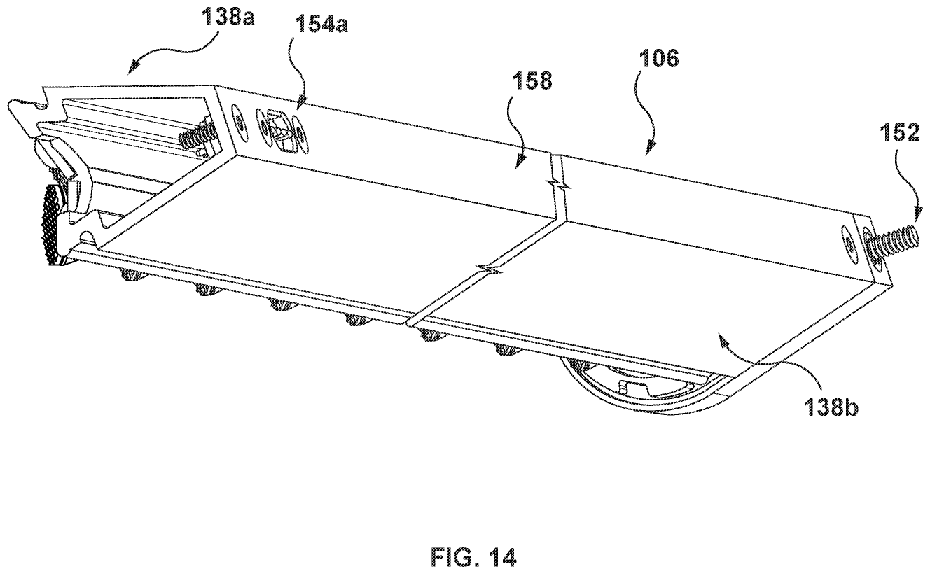

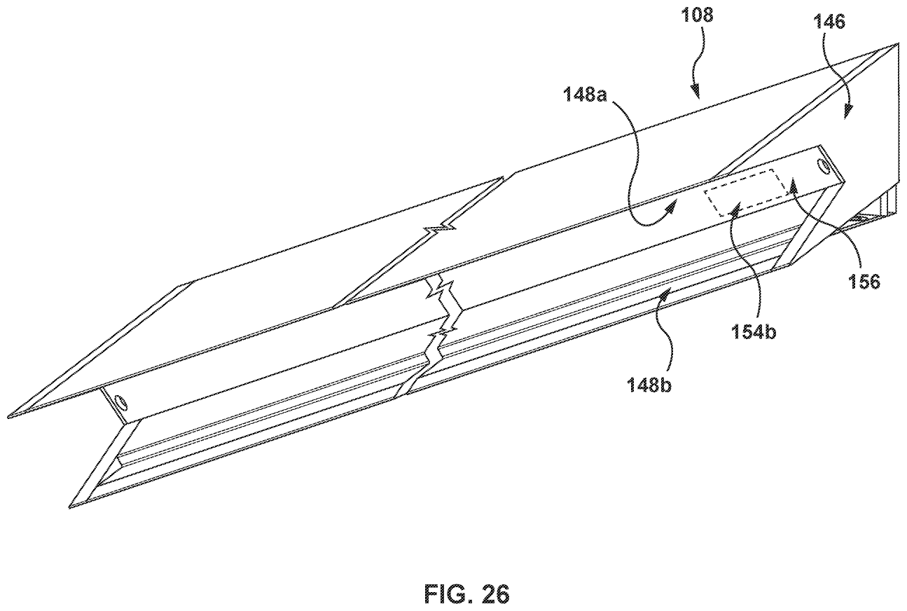

As can be seen in FIGS. 14 and 26, the support structure 108 may include a base structure 146 with a support surface 148b, such as an inclined support surface 148b, to engage the corresponding wall section 138b on the linear housing structure 106. In this case, the base structure 146 may further provide a pair of opposed support surfaces 148a, 148b to engage the corresponding wall sections 138a, 138b, so as to position the linear housing structure into the support structure, for example in a wedge-like configuration, with at least one, in this case a pair of anchor structures shown generally at 152 adjacent opposite end regions of the device 100, to anchor the linear housing structure 106 in the support structure 108 in the wedge-like configuration. Thus, the surfaces 136a, 148a and the surfaces 128b, 148b may provide an effective heat transfer interface for dissipation of heat from the housing structure 106 generated by the first and second arrays of UV LEDs 116, 122, and associated heat generating structures and components therein. In this case, the wedge-like configuration under the forces of anchor structures 52 may provide improved heat transfer in some cases in the heat transfer interface therebetween.

As can be seen in FIG. 14 and schematically in FIG. 26, the linear housing structure 106 and the support structure 108 may further comprise complementary communication nodes 154a, 154b for transferring power and/or communication signals therebetween. For example, the support structure 108 and the linear housing structure 106 may include respective adjacent surfaces 156, 158 in the wedge like configuration with the communication pathways defined thereon.

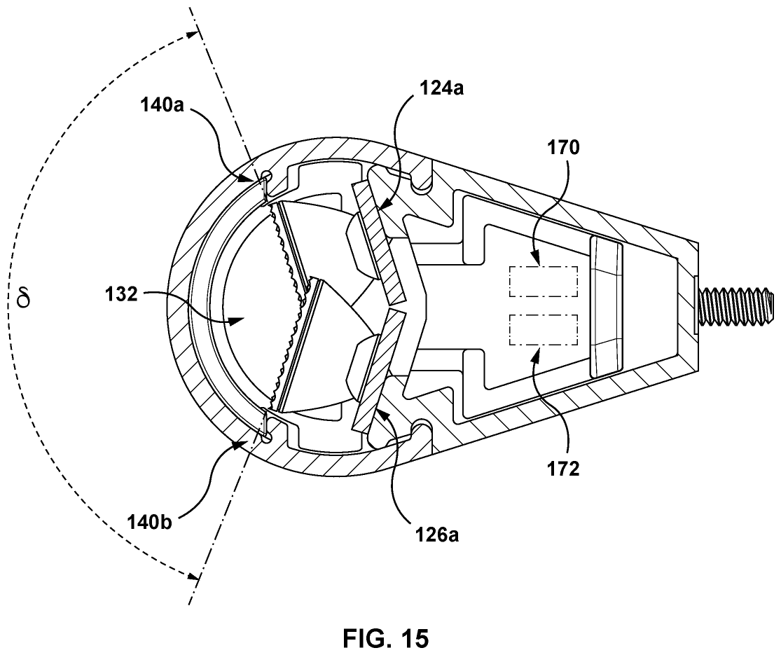

Referring to FIGS. 12 and 15, the linear housing structure 106 may be provided with data storage structure shown schematically at 170 to receive and store data such as authentication data identifying one or more parameters of the linear housing structure, such as a date of fabrication, a change of status of a fault code indicating improper or faulty operation of any of the first or second UV energy emitters, which may be updated by a monitor 172. Similarly, the support structure 108 may be provided with authenticator 174 and a power controller 176, wherein the authenticator may be configured to direct the power controller to deliver power to the first and second arrays when at least one parameter meets a first designated condition, or to interrupt power when at least one parameter meets a second designated condition.

### Clauses

Non-limiting example embodiments are described in the following clauses:

1. A disinfection device for controllably emitting and directing disinfecting UV energy to at least one elongate first target location and at least one second target location in a user space, comprising a housing structure mountable on a support structure and having an outer boundary, wherein the outer boundary is configured to provide:
a first array of outputs to deliver directed disinfecting UV energy to the at least one first target location, with each output of the first array thereof configured to receive directed disinfecting UV energy from one instance of a first array of disinfecting UV energy directors, with each disinfecting director of the first array thereof configured to receive disinfecting UV energy from one instance of a first array of disinfecting UV energy emitters; and
a second array of outputs to deliver directed disinfecting UV energy to the at least one second target location, with each output of the second array thereof configured to receive directed disinfecting UV energy from one instance of a second array of disinfecting UV energy directors, with each disinfecting director of the second array thereof configured to receive disinfecting UV energy from one instance of a second array of disinfecting UV energy emitters.

2. The disinfection device as defined in any of the preceding or following clauses, wherein each instance of the disinfecting UV energy directors of the first array thereof, and the corresponding instance of the disinfecting UV energy emitters of the first array thereof are positioned adjacent a common location on a first mounting surface in the housing structure.

3. The disinfection device as defined in any of the preceding or following clauses, wherein each instance of the disinfecting UV energy directors of the second array thereof, and the corresponding instance of the disinfecting UV energy emitters of the second array thereof are positioned adjacent a common location on a second mounting surface in the housing structure.

4. The disinfection device as defined in any of the preceding or following clauses, wherein the outer boundary of the linear housing structure is defined by an outer boundary wall, with each incidence of the first array of outputs and each incidence of the second array of outputs including a passage through the boundary wall.

5. The disinfection device as defined in any of the preceding or following clauses, wherein the first and second arrays of outputs extend along respective axes, with the axes being substantially parallel or substantially in common.

6. The disinfection device as defined in any of the preceding or following clauses, wherein the outer boundary wall has a continuous or discontinuous cross section.

7. The disinfection device as defined in any of the preceding or following clauses, wherein the outer boundary wall has a curved, linear and/or curvilinear cross section.

8. The disinfection device as defined in any of the preceding or following clauses, wherein the boundary layer has at least one ellipsoidal end region, with a pair of pair of opposed wall sections extending therefrom.

9. The disinfection device as defined in any of the preceding or following clauses, wherein the opposed wall sections are oriented with an acute included angle.

10. The disinfection device as defined in any of the preceding or following clauses, wherein the support structure includes a base with an inclined support surface to engage one of the opposed wall sections.

11. The disinfection device as defined in any of the preceding or following clauses, wherein the support structure further comprises a first power supply segment, and the linear housing structure and support structure are each configured with a first set of contacts for coupling the first power supply segment to a second power supply segment in the linear housing structure to energize the first and second arrays of UV energy emitters.

12. The disinfection device as defined in any of the preceding or following clauses, wherein the first and second arrays of UV energy emitters include UV LEDs, and the second power supply segment includes at least one driver to drive the UV LEDs.

13. The disinfection device as defined in any of the preceding or following clauses, wherein the at least one driver is configured to exchange control data signals with a remote controller networked therewith.

14. The disinfection device as defined in any of the preceding or following clauses, wherein the support structure further comprises a first control data path segment, and the linear housing structure and support structure are each configured with a second set of contacts for coupling the first control data path segment to a second control data path segment in the linear housing structure in communication with the at least one driver.

15. A disinfection device for controllably emitting and directing disinfecting UV energy to at least one first target location and at least one second target location in a user space, comprising a linear housing structure mountable on or in at least one support structure and having an outer boundary, wherein the outer boundary is configured to provide:
at least one output configured:
   to receive a first directed disinfecting UV energy from at least one instance of a first array of disinfecting UV energy directors, with each UV energy disinfecting director of the first array thereof configured to receive a first disinfecting UV energy from one instance of a first array of disinfecting UV energy emitters, and to deliver the first directed disinfecting UV energy to the at least one first target location; and
   to receive a second directed disinfecting UV energy from at least one instance of a second array of disinfecting UV energy directors, with each disinfecting UV energy director of the second array thereof configured to receive a second disinfecting UV energy from one instance of a second array of disinfecting UV energy emitters, and to deliver the second directed disinfecting UV energy to the at least one second target location.

16. The disinfection device as defined in any of the preceding or following clauses, wherein each instance of the disinfecting UV energy directors of the first array thereof, and the corresponding instance of the disinfecting UV energy emitters of the first array thereof are positioned adjacent a common location on a first linear mounting surface in the linear housing structure.

17. The disinfection device as defined in any of the preceding or following clauses, wherein each instance of the disinfecting UV energy directors of the second array thereof, and the corresponding instance of the disinfecting UV energy emitters of the second array thereof are positioned adjacent a common location on a second linear mounting surface in the linear housing structure.

18. The disinfection device as defined in any of the preceding or following clauses, wherein the first and second linear surfaces are defined by corresponding first and second linear LED board structures positioned in the linear housing structure.

19. The disinfection device as defined in any of the preceding or following clauses, wherein the outer boundary of the linear housing structure is defined by an outer boundary wall, with each incidence of the array of at least one output including a passage through the boundary wall.

20. The disinfection device as defined in any of the preceding or following clauses, wherein the outer boundary wall has at least one ellipsoidal end region, with a pair of pair of opposed wall sections extending therefrom.

21. The disinfection device as defined in any of the preceding or following clauses, wherein the at least one passage extends through the at least one ellipsoidal end region along a dimension parallel to a central axis thereof, and a dimension transverse to the central axis.

22. The disinfection device as defined in any of the preceding or following clauses, wherein the at least one passage is defined by a pair of opposed linear boundaries which are spaced by included radial angle therebetween.

23. The disinfection device as defined in any of the preceding or following clauses, wherein the first and second linear LED board structures are each configured to extend along a corresponding linear boundary, so as to cause to the first disinfecting UV energy of the first array to pass through the second directed disinfecting UV energy of the second array.

24. The disinfection device as defined in any of the preceding or following clauses, further comprising at least one UV energy transmissive cover structure to close the at least one passage.

25. The disinfection device as defined in any of the preceding or following clauses, wherein the opposed linear boundaries are oriented with an acute included angle relative to the central axis.

26. The disinfection device as defined in any of the preceding or following clauses, wherein the support structure includes a base structure with a support surface to engage one of the opposed wall sections.

27. The disinfection device as defined in any of the preceding or following clauses, wherein the support structure includes a base with a pair of opposed support surfaces, each to engage a corresponding one of the opposed wall sections, so as to position the linear housing structure into the support structure in a wedge-like configuration.

28. The disinfection device as defined in any of the preceding or following clauses, further comprising at least one anchor structure to anchor the linear housing structure in the support structure in the wedge-like configuration.

29. The disinfection device as defined in any of the preceding or following clauses, wherein the linear housing structure and the support structure further comprise complementary communication pathways for transferring power and/or communication signals therebetween.

30. The disinfection device as defined in any of the preceding or following clauses, wherein the support structure and the linear housing structure include respective adjacent surfaces in the wedge-like configuration with the communication pathways defined thereon.

31. A disinfection device for controllably emitting and directing disinfecting UV energy to at least one first proximal target location defined in a first plane, and at least one second distal target location defined in a second plane, in a user space, comprising a housing structure mountable on or in at least one support structure and having an outer boundary, wherein the outer boundary is configured to provide:
at least one output configured:
   to receive a first directed disinfecting UV energy from a first array of at least one first disinfecting UV engine to deliver the first directed disinfecting UV energy to the at least one first proximal target location; and
   to receive a second directed disinfecting UV energy from a second array of second disinfecting UV engines to deliver the second directed disinfecting UV energy to the at least one second proximal target location.

32. The disinfection device as defined in any of the preceding or following clauses, wherein the first array includes at least one instance of the first disinfecting UV engines, and the second array includes at least two instances of the second disinfecting UV engines.

33. The disinfection device of clause 31, wherein instances of first disinfecting UV engines in the first array are fewer in number than instances of second disinfecting UV engines in the second array, so as to accommodate for a relatively shorter distance for the first directed disinfecting UV energy to travel to the first proximal target location, and the relatively longer distance for the second directed disinfecting UV energy to travel to the second distal target location.

34. The disinfection device as defined in any of the preceding or following clauses, wherein the first array of UV engines are spaced at a first spacing, and the second array of UV engines are spaced at a second spacing, wherein the first spacing is greater than the second spacing.

35. The disinfection device as defined in any of the preceding or following clauses, wherein the second spacing is about twice the first spacing.

36. The disinfection device as defined in any of the preceding or following clauses, the first array and second array are aligned with spaced substantially parallel first and second axes.

37. The disinfection device as defined in any of the preceding or following clauses, wherein the housing structure has opposed outer panel structures configured to engage corresponding opposed inner panels structures in a wedge-like heat transfer configuration.

38. The disinfection device as defined in any of the preceding or following clauses, further comprising at least one anchor structure to anchor the linear housing structure in the support structure so as to establish a heat transfer interface between each outer panel structure and a corresponding inner panel structure.

39. The disinfection device as defined in any of the preceding or following clauses, wherein the outer panel structures are in a wedge-like configuration with the opposed inner panel structures.

40. The disinfection device as defined in any of the preceding or following clauses, wherein the housing structure and the support structure further comprise complementary communication pathways for transferring power and/or communication signals therebetween.

41. The disinfection device as defined in any of the preceding or following clauses, wherein the housing structure comprises data storage structure to receive and store authentication data identifying one or more parameters of the linear housing structure.

42. The disinfection device as defined in any of the preceding or following clauses, wherein the housing structure comprises a monitor to update the one or more parameters.

43. The disinfection device as defined in any of the preceding or following clauses, wherein the support structure comprises an authenticator to communicate with a power controller, wherein the authenticator is configured to direct the power controller to deliver power to the first and second arrays when at least one parameter meets a first designated condition, or to interrupt power when at least one parameter meets a second designated condition.

44. The disinfection device as defined in any of the preceding or following clauses, wherein one or more of the first and second disinfecting UV engines includes a UV energy director which receives UV energy from a UV energy emitter.

45. The disinfection device as defined in any of the preceding or following clauses, wherein the UV energy director includes an optical coupling between a UV energy diffuser structure, a UV energy collimator structure, and the UV energy emitter.

46. The disinfection device as defined in any of the preceding or following clauses, wherein one or more of the first target location, the second target location, the housing structure, the support structure, the first mounting surface, the second mounting surface, the first array, the second array, the support structure, the base structure, the opposed support surfaces, the opposed wall sections, the outer panel structures, the inner panel structures is, or are correspondingly, linear in configuration.

While the present disclosure describes various exemplary embodiments, the disclosure is not so limited. To the contrary, the disclosure is intended to cover various modifications and equivalent arrangements, as will be readily appreciated by the person of ordinary skill in the art.

1.-30. (canceled)

31. A linear disinfection device for controllably emitting and directing disinfecting UV energy to at least one first proximal target location defined in a first plane, and at least one second distal target location defined in a second plane, in a user space, comprising a linear housing structure mountable on or in at least one support structure and having an outer boundary, the linear housing structure containing a first array of first disinfecting UV engines configured to deliver a first directed disinfecting UV energy to the at least one first proximal target location, and a second array of second disinfecting UV engines configured to deliver a second directed disinfecting UV energy to the at least one second proximal target location, wherein the outer boundary is configured to provide
two or more common outputs extending along the linear housing structure, each of the two or more common outputs configured to receive both the first directed disinfecting UV energy from afro the first array of first disinfecting UV engines and a receive the second directed disinfecting UV energy from the second array of second disinfecting UV engines to deliver the first and second directed disinfecting UV energies to the first and second proximal target locations.

32. (canceled)

33. The disinfection device of claim 45, wherein instances of the first disinfecting UV engines in the first array are fewer in number than instances of the second disinfecting UV engines in the second array, so as to enable collective amounts of the first and second directed disinfecting UV energy to be configured by the corresponding numbers of first and second disinfecting UV engines.

34. The disinfection device of claim 33, wherein the first disinfecting UV engines of the first array are spaced at a first spacing, and the second disinfecting UV engines of the second array are spaced at a second spacing, wherein the first spacing is greater than the second spacing, and wherein each of the first disinfecting UV engines is interspersed between a pair of second disinfecting UV engines.

35. The disinfection device of claim 34, wherein the first spacing is about twice the second spacing.

36. The disinfection device of claim 31, wherein the first array and second array are aligned with spaced substantially parallel first and second axes.

37.-39. (canceled)

40. The disinfection device of claim 31, wherein the linear housing structure and the support structure further comprise complementary communication pathways for transferring power and/or communication signals therebetween.

41. The disinfection device of claim 40, wherein the linear housing structure comprises data storage structure to receive and store authentication data identifying one or more parameters of the linear housing structure.

42. The disinfection device of claim 41, wherein the linear housing structure comprises a monitor to update the one or more parameters.

43. The disinfection device of claim 40, wherein the support structure comprises an authenticator to communicate with a power controller, wherein the authenticator is configured to direct the power controller to deliver power to the first and second arrays when at least one parameter meets a first designated condition, or to interrupt power when at least one parameter meets a second designated condition.

44. The disinfection device of claim 31, wherein each of the first and second disinfecting UV engines includes a corresponding first and second UV energy director, each of which receives UV energy from a corresponding first and second UV energy emitter.

45. The disinfection device of claim 44, wherein each of the first and second UV energy directors includes an optical coupling between a corresponding first and second UV energy diffuser structure, a corresponding first and second UV energy collimator structure, and the corresponding first and second UV energy emitter.

46.-47. (canceled)

48. The disinfection device of claim 31, wherein the outer boundary includes an ellipsoidal end region extending along the linear housing structure, with the two or more common outputs in side-by-side relation therein, with opposed outer panel structures extending therefrom.

49. The disinfection device of claim 48, wherein the opposed outer panel structures are oriented at an acute included angle therebetween.

50. The disinfection device of claim 31, wherein the outer boundary of the linear housing structure is defined by an outer boundary wall, with the two or more common outputs defined by a corresponding two or more passages through the boundary wall.

51. The disinfection device of claim 50, wherein the outer boundary wall has at least one ellipsoidal end region, with a pair of opposed outer panel structures extending therefrom.

52. The disinfection device of claim 51, wherein each of the two or more passages extend through the at least one ellipsoidal end region along a dimension parallel to a central axis thereof, and a dimension transverse to the central axis.

53. The disinfection device of claim 52, wherein each of the two or more passages is defined by a pair of opposed linear boundaries which are spaced by an acute included angle therebetween.

54. The disinfection device of claim 34, wherein the first and second disinfecting UV engines include corresponding first and second linear LED board structures, each configured to extend along a corresponding linear boundary, so as to cause to the first disinfecting UV energy of the first array to pass through the second directed disinfecting UV energy of the second array.

55. The disinfection device of claim 53, further comprising at least one UV energy transmissive cover structure to close each corresponding passage.

56. The disinfection device of claim 55, wherein the opposed linear boundaries are oriented with an acute included angle relative to the central axis.
